(19) United States
(12) Patent Application Publication
(10) Pub. No.: US 2014/0295435 A1
(43) Pub. Date: Oct. 2, 2014

(54) METHOD FOR DETECTING BACILLUS ANTHRACIS

(71) Applicant: GeneReach Biotechnology Corp., Taichung City (TW)

(72) Inventors: Chen SU, Taichung City (TW); Hwa-Tang WANG, Taichung City (TW); Hsiao-Fen CHANG, Taichung City (TW); Yun Lung TSAI, Taichung City (TW); Li Juan MA, Taichung City (TW); Shih-Han WENG, Taichung City (TW)

(73) Assignee: GeneReach Biotechnology Corp., Taichung City (TW)

(21) Appl. No.: 14/015,330

(22) Filed: Aug. 30, 2013

(30) Foreign Application Priority Data
Apr. 2, 2013 (TW) 102111848

(51) Int. Cl. C12Q 1/68

(52) U.S. Cl. CPC C12Q 1/689
USPC 435/6.12

(57) ABSTRACT

A method for detecting Bacillus Anthracis comprises steps: obtaining DNA of a sample; respectively mixing the DNA with Primer Sets pXO1, pXO2 and PL3 to form a first reactant mixture, a second reactant mixture and a third reactant mixture, wherein the Primer Sets pXO1, pXO2 and PL3 are respectively sequences specially designed for pXO1, pXO2 and PL3; respectively undertaking PCRs of the first reactant mixture, the second reactant mixture and the third reactant mixture; and detecting whether the sample contains sequences of pXO1, pXO2 and PL3 simultaneously to determine whether the sample contains Bacillus Anthracis. The present invention detects whether the sample contains pXO1, pXO2 and PL3 simultaneously to determine whether the sample contains Bacillus Anthracis and further uses specified primer sets to undertake PCRs and increase the sensitivity and specificity of detection. Thereby is increased the speed and accuracy of detection.

### FIELD OF THE INVENTION

The present invention relates to a biochemical test method, particularly to a method for detecting Bacillus Anthracis.

### BACKGROUND OF THE INVENTION

Bacillus Anthracis may be used as a biological weapon. Terrorists usually parcel and mail it to infect people and cause tenor to people. As test of Bacillus Anthracis consumes a longer period of time, it normally has spread out before its existence is confirmed. Therefore, how to detect Bacillus Anthracis fast and accurately has been a target the researchers are devoted to.

At present, there have been many gene molecule-based test technologies able to detect microorganisms fast, and PCR (Polymerase Chain Reaction) is the most frequently-used among them. A PCR test includes three phases: denaturation, primer annealing, and extension, which are respectively undertaken at different temperatures. The current PCR test needs the following materials or reagents: a DNA (deoxyribonucleic acid) template the test intends to extend; oligonucleotide primer pairs complementary to specified sequences on each strand of the DNA template; thermally-stable DNA polymerases; and dNTP (deoxynucleotide triphosphate). A PCR apparatus heats and cools the reaction sample repeatedly to make the reaction sample experience three different temperatures cyclically and thus extends specified regions of the DNA template.

The first phase of PCR undertakes a denaturation reaction: heat the reaction sample to a high temperature, preferably a temperature of 90-95° C., to separate the double-strand template DNA into single-strand DNAs.

The second phase of PCR is a primer annealing reaction: cool the single-stand DNAs to a lower temperature to make the primers combine with the single-strand DNAs to form composites of primers and single-strand DNAs; the temperature of primer annealing is dependent on the melting temperature (Tm) of the primer, normally within 35-65° C.

The third phase of PCR undertakes an extension reaction: maintain the composites of the primers and the single-strand DNAs at an appropriate temperature, typically 72° C., and use the DNA polymerase to extend the primers and form new single-strand DNAs respectively complementary to the strands of the template DNA.

Each cycle of the three phases can double the template DNA. Millions of duplicates of the template DNA will be generated after the cycle of the three phases of denaturation, primer annealing and extension has been repeated for about 20-40 times.

Although PCR can effectively duplicate genes and promote test accuracy, it can only be operated by professional personnel in a laboratory. Therefore, the current technologies are hard to detect Bacillus Anthracis effectively and instantly.

### SUMMARY OF THE INVENTION

The primary objective of the present invention is to overcome the problem that the conventional technologies cannot fast and accurately detect Bacillus Anthracis.

To achieve the abovementioned objective, the present invention proposes a method for detecting Bacillus Anthracis, which comprises

Step S1: obtaining DNA of a sample;

Step S2: respectively mixing the DNA with Primer Sets pXO1, pXO2 and PL3 to form a first reactant mixture, a second reactant mixture and a third reactant mixture, wherein

the sequence of the forward primer of pXO1 is

GGACACATACTAGTGAAGTACATGGAA (SEQ ID NO: 1);

the sequence of the reverse primer of pXO1 is

TCCTGCAGATACACTCCCACCAA (SEQ ID NO: 2);

the sequence of the forward primer of pXO2 is

TCTTCCCAGATAATGCATCGCT (SEQ ID NO:

the sequence of the reverse primer of pXO2 is

CACGGAATGCTGTTTCCTCAT (SEQ ID NO: 4);

the sequence of the forward primer of PL3 is

CGATTGATGAAGGCGACAATGTACT (SEQ ID NO: 5);

the sequence of the reverse primer of PL3 is

CTCCTCGTGTGGATCGGTTGTTT (SEQ ID NO: 6);

Step S3: respectively undertaking PCRs of the first, second and third reactant mixtures; and

Step S4: detecting whether the first, second and third reactant mixtures respectively have pXO1, pXO2 and PL3 to determine whether the sample contains i Bacillus Anthracis.

The present invention has the following features:

1. Determining whether the sample contains the virulent Bacillus Anthracis via detecting whether the sample has pXO1, pXO2 and PL3 simultaneously; and

2. Using the specified primer sets of pXO1, pXO2 and PL3 to increase the sensitivity and specificity of the test.

### BRIEF DESCRIPTION OF THE DRAWINGS

FIG. 1 shows a flowchart of a method for detecting Bacillus Anthracis according to one embodiment of the present invention;

FIG. 2 is a perspective view of an apparatus according to one embodiment he present invention;

FIG. 3 is a partial exploded view of an apparatus according to one embodiment of the present invention; and

FIG. 4 is a partial sectional view of an apparatus according to one embodiment of the present invention.

### DETAILED DESCRIPTION OF THE PREFERRED EMBODIMENTS

The technical contents of the present invention are described in detail in cooperation with the drawings below.

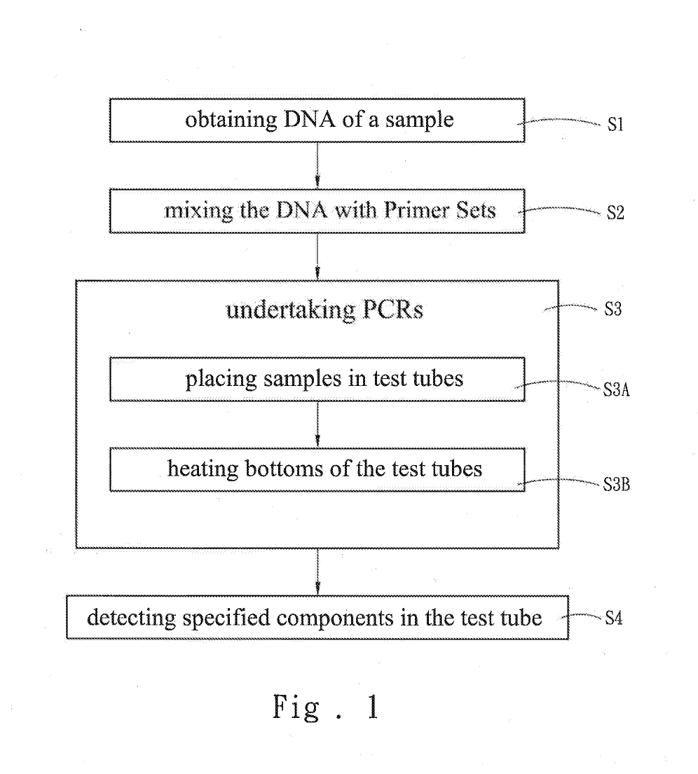

Refer to FIG. 1. The present invention proposes a method for detecting Bacillus Anthracis, which comprises Steps S1-S4.

In Step S1, obtain DNA (deoxyribonucleic acid) of a sample.

In Step S2, respectively mix the DNA with Primer Sets pXO1, pXO2 and PL3 to form a first reactant mixture, a second reactant mixture and a third reactant mixture, wherein

the sequence of the forward primer of pXO1 is

GGACACATACTAGTGAAGTACATGGAA (SEQ ID NO: 1);

the sequence of the reverse primer of pXO1 is

TCCTGCAGATACACTCCCACCAA (SEQ ID NO: 2);

the sequence of the forward primer of pXO2 is

TCTTCCCAGATAATGCATCGCT (SEQ ID NO: 3);

the sequence of the reverse primer of pXO2 is

CACGGAATGCTGTTTCCTCAT (SEQ :ID NO: 4);

the sequence of the forward primer of PL3 is

CGATTGATGAAGGCGACAATGTACT (SEQ ID NO: 5);

the sequence of the reverse primer of PL3 is

CTCCTCGTGTGGATCGGTTGTTT (SEQ ID NO: 6).

The forward primers and the reverse primers function as the initiation points to facilitate the duplication of DNA. The sequences of the forward primers and the reverse primers exist between the terminal 5′ and the terminal 3′. In the specification, the RT-PCR (Real Time-PCR) of a probe system is used as the exemplification, wherein

the probe of pXO1 is

FAM—AGTGCATGCGTCGTTCT—MGB (SEQ ID NO: 7), and

the probe of pXO2 is

VIC—TCCCAAGAGCCTCTG—MGB (SEQ ID NO: 8), and

the probe of PL3 is

FAM—AGTGCATGCGTCGTTCT—MGB (SEQ ID NO: 7).

Each probe contains a reporter dye and a quencher dye respectively appearing at the front and rear of the sequence thereof. For example, “FAM” and “MGB” are respectively the reporter dye and the quencher dye in the probe of pXO1. While the probe has not adhered to a target product yet, the quencher dye is closer to the reporter dye and able to absorb the fluorescence of the reporter dye. While the probe finds the target product, i.e. meets the complementary DNA, the probe is hydrolyzed. Thus, the reporter dye is separated from the quencher dye and presents the fluorescence thereof. Then, the observer can perceive the fluorescence.

In Step S, respectively undertake PCRs of the first, second and third reactant mixtures. Below is introduced a method able to fast and effectively undertake PCR, which comprises Step S3A and Step S3B.

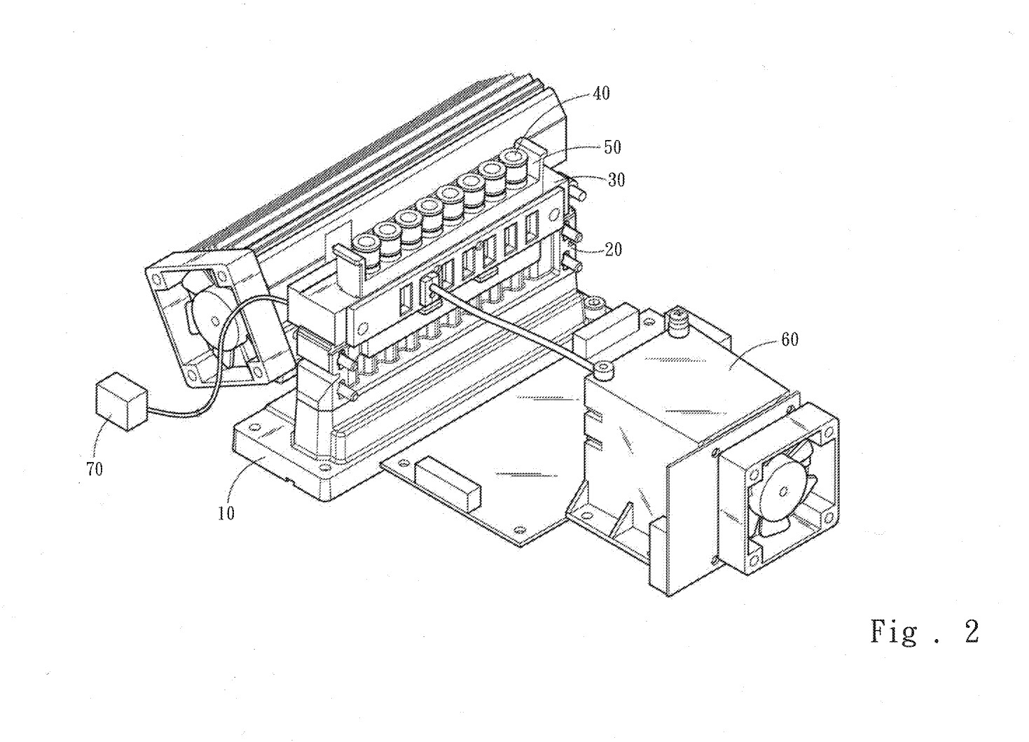

In Step S3A, place the samples in test tubes 40, wherein the first, second and third reactant mixtures are respectively placed in three different test tubes 40 (shown in FIG. 2). The test tubes 40 are all form of long tubes. in this embodiment, the first, second and third reactant mixtures are respectively placed in different test tubes 40, However, the present invention does not constrain that the first, second and third reactant mixtures must be respectively placed in different test tubes 40. In the present invention, the first, second and third reactant mixtures may be all placed in an identical test tube 40 for reaction and detection.

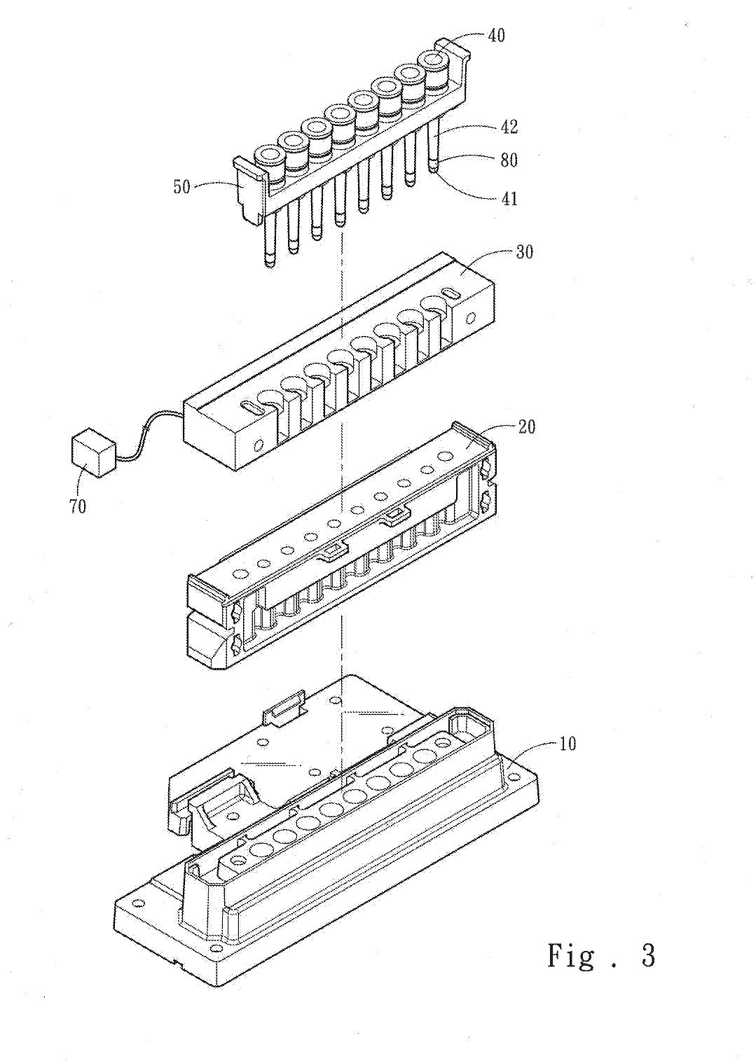

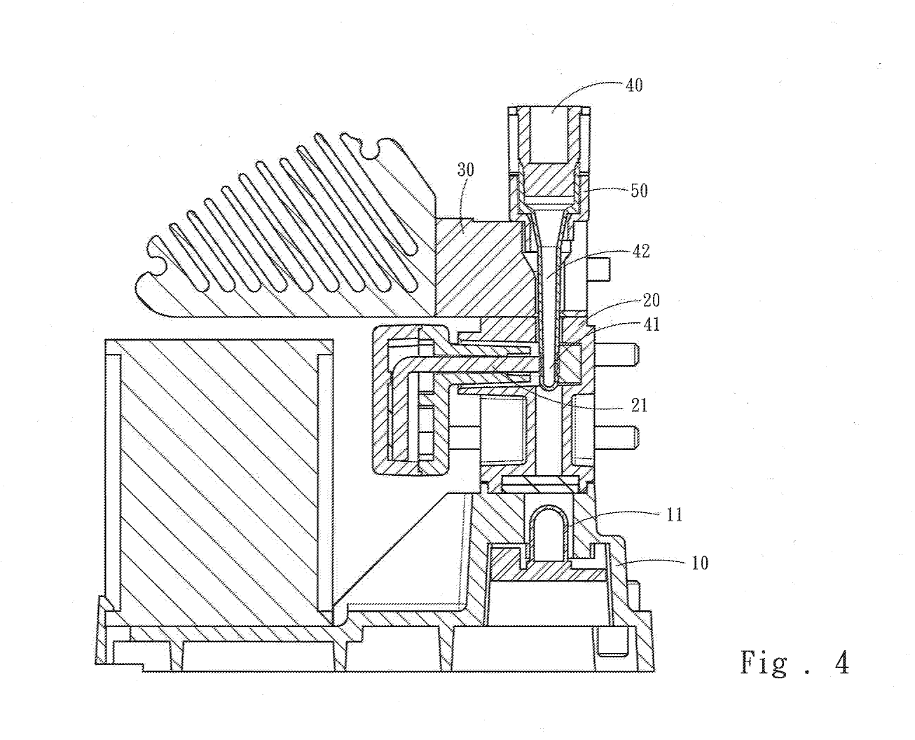

In Step S3B, heat the bottoms of the test tubes 40 to undertake iiPCRs (insulated isothermal PCR.) of the first, second and third reactant mixtures. Refer to FIGS. 2-4 for the apparatus used by the present invention. The apparatus comprises a base 10, a heater 20 connecting with the base 10, an auxiliary heater 30 connecting with the heater 20, at least one test tube 40, a tube frame 50 fixing the test tubes 40 to the auxiliary heater 30, and a observation module 60. The base 10 includes a light source 11 (a light emitting diode in this embodiment) for illuminating the test tubes 40. The heater 20 includes a primary heating element 21 for, heating the bottoms of the test tubes 40 to a denaturation temperature within 90-98° C. and facilitating PCR. As the test tube 40 is in form of a long tube, the middle region and the higher region of the test tube 40 have lower temperature. Thus, the primer annealing reaction and the extension reaction take place in the higher region and the middle region of the test tube 40. Then, three phases of PCR are undertaken in an single test tube 40. In order to transfer heat to the test tube 40 more accurately, the bottom 41 of the test tube 40 is surrounded by a high thermal conductivity element 80. Thereby, the primary heating element 21 contacts the test tube 40 through the high thermal conductivity element 80. In order to control the temperature variation of the test tube 40 more effectively, a temperature control device 70 is coupled to the auxiliary heater 30. The auxiliary heater 30 is spaced from the primary heater 20 by a given distance and corresponding to the middle region 42 of the test tube 40. The auxiliary heater 30 does not, contact the test tube 40. The auxiliary heater 30 stabilizes the temperature of the middle region 42 of the test tube 40 via controlling the temperature of the environment around the test tube 40. The auxiliary heater 30 undertakes an auxiliary temperature control at the middle region 42 of the test tube 40 and heats the test tube 40 to an auxiliary heating temperature, which is lower than the denaturation temperature and within 40-50° C., so as to stabilize the progress of PCR.

In Step S4, detect the components. If it is detected that the first, second and third reactant mixtures respectively contain the components of pXO1, pXO2 and PL3, the sample is determined to have Bacillus Anthracis. For example, while the DNA contains pXO1, the probe of pXO1 is hydrolyzed to generate fluorescence. During the process of PCR, pXO1 is duplicated persistently, and the fluorescence becomes more and more obvious. The observers can determine whether the sample contains pXO1 according to the status of the fluorescence. Whether the sample contains pXO2 or PL3 is also detected in the similar way. The sample would not contain the virulent Bacillus Anthracis unless the sample contains pXO1, pXO2 and PL3 simultaneously.

In conclusion, the present invention is characterized in

1. Determining whether the sample contains the virulent Bacillus Anthracis via detecting whether the sample has pXO1, pXO2 and PL3 simultaneously;

2: Using the specified primer sets of pXO1, pXO2 and PL3 to increase the sensitivity and specificity of the test; and

3. Undertaking detection with iiPCR to increase the speed and utility of detection.

1. A method for detecting Bacillus Anthracis, comprising
Step S1 obtaining DNA of a sample;
Step S2: respectively mixing the DNA with Primer Sets pXO1, pXO2 and PL3 to form a first reactant mixture, a second reactant mixture and a third reactant mixture, wherein
a sequence of a forward primer of pXO1 is
GGACACATACTAGTGAAGTACATGGAA (SEQ ID NO: 1);
a sequence of a reverse primer of pXO1 is
TCCTGCAGATACACTCCCACCAA (SEQ ID NO: 2);
a sequence of a forward primer of pXO2 is
TCTTCCCAGATAATGCATCGCT (SEQ ID NO: 3);
a sequence of a reverse primer of pXO2 is
CACGGAATGCTGTTTCCTCAT (SEQ ID NO: 4);
a sequence of a forward primer of PL3 is
CGATTGATGAAGGCGACAATGTACT (SEQ ID NO: 5);
a sequence of a reverse primer of PL3 is
CTCCTCGTGTGGATCGGTTGTTT (SEQ ID NO: 6);
Step S3: respectively undertaking polymerase chain reactions (PCRs) of the first reactant mixture, the second reactant mixture and the third reactant mixture; and
Step S4: detecting the first reactant mixture, the second reactant mixture and the third reactant mixture, and determining that the sample contains Bacillus Anthracis if the first reactant mixture, the second reactant mixture and the third reactant mixture respectively contain components of pXO1, pXO2 and PL3.

2. The method for detecting Bacillus Anthracis according to claim 1, wherein in Step S2,
a probe of pXO1 is
FAM—AGTGCATGCGTCGTTCT—MGB (SEQ ID NO: 7), and
a probe of pXO2 is
VIC—TCCCAAGAGCCTCTG—MGB (SEQ ID NO: 8), and
a probe of PL3 is
FAM—AGTGCATGCGTCGTTCT—MGB (SEQ ID NO: 7), and
wherein in Step S3, a real-time PCR of a probe system is undertaken.

3. The method for detecting Bacillus Anthracis according to claim 2, wherein Step S3 further comprises:
Step S3A: respectively placing the first reactant mixture, the second reactant mixture and the third reactant mixture in three different test tubes, wherein the test tubes are all In form of long tubes; and
Step S3B: heating bottoms of the test tubes to undertake PCRs of the first reactant mixture, the second reactant mixture and the third reactant mixture.

4. The method for detecting Bacillus Anthracis according to claim 3, wherein in Step S3B, the bottoms of the test tubes is heated to a denaturation temperature and maintained at the denaturation temperature by a primary heating element.

5. The method for detecting Bacillus Anthracis according to claim 4, wherein the denaturation temperature is within 90-98° C.

6. The method for detecting Bacillus Anthracis according to claim 4, wherein in Step S3B, a middle region of the test tube is heated by an auxiliary heater to an auxiliary heating temperature, and wherein the auxiliary heating temperature is lower than the denaturation temperature.

7. The method for detecting Bacillus Anthracis according to claim 6, wherein the auxiliary heating temperature is within 40-50° C.


SEQUENCE LISTING (8 sequences)

SEQ ID NO: 1 - DNA, 27 bp, Artificial Sequence
  Synthetic DNA Primer
ggacacatactagtgaagtacatggaa

SEQ ID NO: 2 - DNA, 23 bp, Artificial Sequence
  Synthetic DNA Primer
tcctgcagatacactcccaccaa

SEQ ID NO: 3 - DNA, 22 bp, Artificial Sequence
  Synthetic DNA Primer
tcttcccagataatgcatcgct

SEQ ID NO: 4 - DNA, 21 bp, Artificial Sequence
  Synthetic DNA Primer
cacggaatgctgtttcctcat

SEQ ID NO: 5 - DNA, 25 bp, Artificial Sequence
  Synthetic DNA Primer
cgattgatgaaggcgacaatgtact

SEQ ID NO: 6 - DNA, 23 bp, Artificial Sequence
  Synthetic DNA Primer
ctcctcgtgtggatcggttgttt

SEQ ID NO: 7 - DNA, 17 bp, Artificial Sequence
  Synthetic DNA Probe
agtgcatgcgtcgttct

SEQ ID NO: 8 - DNA, 15 bp, Artificial Sequence
  Synthetic DNA Probe
tcccaagagcctctg